(19) United States
(12) Patent Application Publication
(10) Pub. No.: US 2024/0293395 A1
(43) Pub. Date: Sep. 5, 2024

(54) NALTREXONE FOR IMPROVING THE EFFECTIVENESS OF 5-HT RECEPTOR SUBTYPE 2A, 2B or 2C AGONISTS

(71) Applicant: LDN Pharma Limited, London (GB)

(72) Inventors: Wai Liu, London (GB); Ian Thompson, London (GB); Francis Hood, London (GB)

(21) Appl. No.: 18/573,359

(22) PCT Filed: Jun. 23, 2022
(86) PCT No.: PCT/GB2022/051608

§ 371 Date: Dec. 21, 2023

(30) Foreign Application Priority Data
Jun. 23, 2021 (GB) 2109022.0

(51) Int. Cl. A61K 31/485; A61K 31/135; A61K 31/36; A61K 31/48; A61K 31/675

(52) U.S. Cl. CPC A61K 31/485; A61K 31/135; A61K 31/36; A61K 31/48; A61K 31/675

(57) ABSTRACT

A composition comprising an agonist of a 5-hydroxytryptamine (5-HT) receptor subtype 2A, 2B or 2C (5-HT2A, 5-HT2B or 5-HT2C), for the use in the treatment of a cognitive or neurological disorder in a patient, said patient having been pre-treated with naltrexone, or a metabolite thereof or an analogue selected from the group consisting of methylnaltrexone, nalmefene and nalorphine.

### FIELD OF THE INVENTION

The invention relates to compositions for use in the treatment of a cognitive or neurological disorder in a patient.

### BACKGROUND OF THE INVENTION

Serotonin (5-hydroxytryptamine; 5-HT) is a biogenic amine with a complex and multifaceted biological function. The main receptors and their subtypes are 5-HT1 (5-HT1A, 5-HT1B, 5-HT1D, 5-HT1E and 5-HT1F), 5-HT2 (5-HT2A, 5-HT2B and 5-HT2C), 5-HT3, 5-HT4, 5-HT5 (5-HT5A, 5-HT5B), 5-HT6 and 5-HT7. The receptors are all G-protein coupled receptors (GPCR) except 5-HT3 receptors, which are ionic channels.

Basic Neurochemistry: Molecular, Cellular and Medical Aspects. 6th edition; Chapter: Serotonin Receptors discloses that a high density of 5-HT2A receptors is found in many cortical areas. These receptors are particularly concentrated in the frontal cortex. 5-HT2A receptors also are found in high density in the claustrum, a region which is connected to the visual cortex; in parts of the limbic system; and in the basal ganglia and the olfactory nuclei.

5-HT2B receptors are found in the central nervous system, such as the subiculum, the substantia nigra and the peripheral nervous system, such as vascular smooth muscle.

5-HT2C receptors are present in high density in the choroid plexus. It has been proposed that 5-HT-induced activation of 5-HT2C receptors could regulate the composition and volume of the cerebrospinal fluid. 5-HT2C receptors also are found throughout the brain, particularly in areas of the limbic system, including the hypothalamus, hippocampus, septum, neocortex and regions associated with motor behaviour, including the substantia nigra and globus pallidus.

Drugs which are capable of either selectively stimulating or inhibiting these receptor subtypes have therapeutic potential in a wide variety of disease conditions. Specifically, agonists of 5-HT2 receptors are known to be useful in the treatment of various neurological and cognitive disorders, such as Alzheimer's disease, dementia, motor neuron disease (MND), depression, psychosis and anxiety.

For example, Meltzer et. al, Curr Pharm Biotechnol. (2012), 13(8): “Serotonin receptors as targets for drugs useful to treat psychosis and cognitive impairment in schizophrenia” discloses the use of 5-HT2C agonists in the treatment of psychosis and cognitive impairment.

Lin et. al. Clin Psychopharmacol Neurosci. (2014), 12(3), 196-202; “Serotonin and Mental Disorders: A Concise Review on Molecular Neuroimaging Evidence” discusses the impact of the serotonergic system on mental disorders.

Terry et. al. Behavioural Brain Research (2008) 195(1), 30-38; “Cognitive dysfunction in neuropsychiatric disorders: Selected serotonin receptor subtypes as therapeutic targets” explains that serotonin plays a key role in mood and emotion, and a number of psychiatric conditions including anxiety, depression, schizophrenia, and anorexia nervosa. It also highlights the importance of 5-HT2 receptors in studies related to cognitive function.

Cowen et. al., Journal of Psychopharmacology (2013); “The role of serotonin in cognitive function: evidence from recent studies and implications for understanding depression” discloses that agonism at specific 5-HT receptors can exert effects in animal models of cognition.

There exists a need in the field to improve the therapeutic efficacy of such agonists of 5-HT2 receptors, in order to improve treatment of patients with neurological and cognitive disorders.

### SUMMARY OF THE INVENTION

The inventors of the present invention have discovered that naltrexone, an orally-administered opioid antagonist with the chemical name morphinan-6-one, 17-(cyclopropylmethyl)-4,5-epoxy-3,14-dihydroxy-(5a), selectively increases serotonin (5-HT) receptor 2 levels and can therefore be used to aid the effectiveness of agonists of 5-HT2 receptors. This has been demonstrated in the examples of the present application where it can be seen that both higher dose and low dose naltrexone increase the levels of mRNA for one or more of 5-HT2A, 5-HT2B or 5-HT2C receptor proteins.

In a first aspect of the invention, there is provided a composition comprising an agonist of a 5-hydroxytryptamine (5-HT) receptor subtype 2A, 2B or 2C (5-HT2A, 5-HT2B or 5-HT2C), for the use in the treatment of a cognitive or neurological disorder in a patient, said patient having been pre-treated with naltrexone, or a metabolite thereof or an analogue selected from the group consisting of methylnaltrexone, nalmefene and nalorphine.

In a second aspect of the invention, there is provided a composition comprising naltrexone, or a metabolite thereof, or an analogue selected from the group consisting of methylnaltrexone, nalmefene and nalorphine, for use in the treatment of a cognitive or neurological disorder in a patient, wherein said patient is also being administered or intended to be administered an agonist of a 5-hydroxytryptamine receptor subtype 2A, 2B or 2C (5-HT2A, 5-HT2B or 5-HT2C).

In a third aspect of the invention, there is provided a composition comprising naltrexone, or a metabolite thereof, or an analogue selected from the group consisting of methylnaltrexone, nalmefene and nalorphine, for separate, simultaneous or sequential administration with an agonist of a 5-hydroxytryptamine receptor subtype 2A, 2B or 2C (5-HT2A, 5-HT2B or 5-HT2C), for the treatment of a cognitive or neurological disorder.

### DETAILED DESCRIPTION OF THE INVENTION

According to a first aspect of the invention, there is provided a composition comprising an agonist of a 5-hydroxytryptamine (5-HT) receptor subtype 2A, 2B or 2C (5-HT2A, 5-HT2B or 5-HT2C), for the use in the treatment of a cognitive or neurological disorder in a patient, said patient having been pre-treated with naltrexone, or a metabolite thereof or an analogue selected from the group consisting of methylnaltrexone, nalmefene and nalorphine.

As used herein, the term “agonist” has its conventional meaning as used in the art. Agonists of the 5-hydroxytryptamine (5-HT) receptor subtype 2A, 2B or 2C are compounds that activate 5-HT2A, 5-HT2B or 5-HT2C receptors. These are the three subtypes that belong to the serotonin 5-HT2 receptor subfamily. An agonist of a 5-HT2A, 5-HT2B or 5-HT2C receptor is a full or partial agonist of one or more of the 5-HT2A, 5-HT2B or 5-HT2C receptors.

As used herein the terms “cognitive disorder” or “neurological disorder” have their conventional meanings in the art. Cognitive disorders are a category of mental health disorders that primarily affect cognitive abilities including learning, memory, perception, and problem solving. Neurological disorders are diseases of the central and peripheral nervous system.

As used herein, the term “subject” refers to any animal (for example, a mammal), including, but not limited to, humans, non-human primates, canines, felines, rodents, and the like, which is to be the recipient of a treatment in which a composition comprising an agonist of the 5-hydroxytryptamine (5-HT) receptor subtype 2A, 2B or 2C (5-HT2A, 5-HT2B or 5-HT2C) is to be used according to the present invention. Typically, the terms “subject” and “patient” are used interchangeably herein in reference to a human subject.

As used herein “naltrexone” refers to morphinan-6-one, 17-(cyclopropylmethyl)-4,5-epoxy-3,14-dihydroxy-(5α) and pharmaceutically acceptable salts, solvates, hydrates, stereoisomers, clathrates and prodrugs thereof. Its empirical formula is C₂₀H₂₃NO₄ and its molecular weight is 341.41 in the anhydrous form (<1% maximum water content). The chemical structure of naltrexone is:

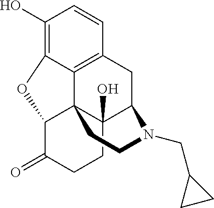

The present invention also encompasses metabolites of naltrexone, such as 6 beta-naltrexol; 2-hydroxyl-3-methoxyl-6-beta-naltrexol (HMN); 2-hydroxy-3-methoxynaltrexone; noroxymorphone; and 3-methoxy-6-beta-naltrexol.

The selected analogues methylnaltrexone, nalmefene and nalorphine are also encompassed in the invention.

The term “pre-treated” has its conventional meaning, that is, prior to being administered the agonist of the 5-hydroxytryptamine (5-HT) receptor subtype 2A, 2B or 2C (5-HT2A, 5-HT2B or 5-HT2C), the patient has been administered with naltrexone, or a metabolite or analogue thereof.

In a second aspect of the invention, there is provided a composition comprising naltrexone, or a metabolite thereof, or an analogue selected from the group consisting of methylnaltrexone, nalmefene and nalorphine, for use in the treatment of a cognitive or neurological disorder in a patient, wherein said patient is also being administered or intended to be administered an agonist of a 5-hydroxytryptamine receptor subtype 2A, 2B or 2C (5-HT2A, 5-HT2B or 5-HT2C).

In a third aspect of the invention there is provided a composition comprising naltrexone, or a metabolite thereof, or an analogue selected from the group consisting of methylnaltrexone, nalmefene and nalorphine, for separate, simultaneous or sequential administration with an agonist of a 5-hydroxytryptamine receptor subtype 2A, 2B or 2C (5-HT2A, 5-HT2B or 5-HT2C), for the treatment of a cognitive or neurological disorder.

As used herein, the term “treatment” refers to the therapeutic measures that cure, slow down, and/or halt progression of a diagnosed pathologic condition or disorder.

In certain embodiments, the agonist may be administered simultaneously, separately, or sequentially alongside naltrexone or a metabolite or analogue thereof.

As used herein, the terms “concurrent administration” or “concurrently” or “simultaneous”, “sequential” or “separate” mean that administration of the agonist and naltrexone or a metabolite or analogue thereof occur as part of the same treatment regimen.

“Simultaneous” administration, as defined herein, includes the administration of the agonist and naltrexone or a metabolite or analogue thereof within about 2 hours or about 1 hour or less of each other, even more preferably at the same time.

“Separate” administration, as defined herein, includes the administration of the agonist and naltrexone or a metabolite or analogue thereof, more than about 12 hours, or about 8 hours, or about 6 hours or about 4 hours or about 2 hours apart.

“Sequential” administration, as defined herein, includes the administration of the agonist of a 5-HT2A, 5-HT2B or 5-HT2C receptor and naltrexone, or a metabolite or analogue thereof, each in multiple aliquots and/or doses and/or on separate occasions. The agonist of a 5-HT2A, 5-HT2B or 5-HT2C receptor may be administered to the subject before or after administration of naltrexone or a metabolite thereof.

In a preferred embodiment, the naltrexone, or metabolite or analogue thereof, is to be administered prior to the administration of the agonist of a 5-HT2A, 5-HT2B or 5-HT2C receptor, preferably between 1 to 4 days before administration of the agonist of a 5-HT2A, 5-HT2B or 5-HT2C receptor.

In a preferred embodiment, the naltrexone or metabolite or analogue thereof is in a dosage amount of 0.01 mg to 50 mg, more preferably 1 mg to 10 mg. In a preferred embodiment, the dosage amount 3 mg to 4.5 mg (i.e. a low dosage). In another preferred embodiment, the dosage amount is 5 mg to 10 mg (i.e. a higher dosage amount).

In a preferred embodiment, the agonist of a 5-HT2A, 5-HT2B or 5-HT2C receptor is selected from the group consisting of psilocybin, psilocin, mescaline, lysergic acid diethylamide (LSD), ketamine, salvinorin A, ibotenic acid, muscimol, N, N-dimethyltryptamine (DMT), 3,4-methylenedioxymethamphetamine (MDMA), methyldiethanolamine, also known as N-methyl diethanolamine (MDEA), 3,4-methylenedioxy amphetamine (MDA), 4-Bromo-2,5-dimethoxyphenethyl-amine (2C-B); 1-(8-Bromo-2,3,6,7-tetra-hydrobenzo[1,2-b;4,5-b′]difuran-4-yl)2-amino-ethane (2C-B-fly); 4-Ethyl-2,5-dimethoxyphenethylamine (2C-E); 4-Ethyl-thio-2,5-dimethoxy phenethylamine (2C-T-2); 4-Ethylthio-2,5-dimethoxy-amphetamine (ALEPH-2); 4-Ethylthio-2,5-dimethoxyphenylbutylamine (4C-T-2); 2,5-Dimethoxy-4-ethoxyamphetamine (MEM); 2,4,5-Trimethox-amphetamine (TMA-2); 3,4,5-Trimethoxamphetamine (TMA); 2,5-Dimethoxy-4-bromo-amphetamine (DOB); 2,5-Dimethoxy-4-iodo-amphetamine (DOI); 2,5-Dimethoxy-4-methylamphetamine (DOM); 2,5-Dimethoxy-4-ethylamphetamine (DOET); 5-Methoxy-N, N-dimethyl-tryptamine (5-MeO-DMT); N,N-Dipropyltryptamine (DPT); 5-Methoxy-N-methyl-N-isopropyltryptamine (5-MeO-MIPT); N,N-Diisopropyltryptamine (DIPT); 5-Methoxy-N,N-diiso-propyltryptamine (5-MeO-DIPT); 6-Fluoro-N,N-dimethyltryptamine (6-fluoro-DMT); lisuride; ibogaine; cis-2a; RR-2b; SS-2c; 2-Ethyl-5-methoxy-N,N-dimethyltryptamine (EMDT), serotonin hydrochloride, m-chlorophenylbiguanide hydrochloride, N-methylquipazine dimaleate, PSEM 895, quipazine dimaleate, RS56812 hydrochloride, serotonin hydrochloride, SR7227 hydrochloride, 1-phenylbiguanide hydrochloride, cereulide, 2-methyl-5-HT, alpha-methyltryptamine, bufotenin, chlorophenylbiguanide, ibogaine, varenicline, YM-31636, or metabolites thereof and combinations thereof. More preferably the agonist of a 5-HT2A, 5-HT2B or 5-HT2C receptor is psilocybin, ketamine, MDMA, LSD and the metabolites thereof.

In the present invention, the agonist of a 5-HT2A, 5-HT2B or 5-HT2C receptor may be a serotonin analogue. By “serotonin analogue” it is meant a functional analogue of serotonin i.e. compounds showing chemical and biological similarity to serotonin. This may be achieved by making modifications to serotonin in order to prepare a new molecule with similar chemical and biological properties.

In a preferred embodiment, the agonist of a 5-HT2A, 5-HT2B or 5-HT2C receptor is to be administered at its approved therapeutic dose. An approved therapeutic dose will be apparent to one skilled in the art, and may vary according to age, sex, weight, which the skilled person is capable of determining.

In a preferred embodiment, the cognitive or neurological disorder is Alzheimer's disease, dementia, motor neuron disease (MND), depression, psychosis, anxiety, obsessive compulsive disorder (OCD), post-traumatic stress disorder (PTSD), paranoia, panic attacks or flashbacks.

In a preferred embodiment the naltrexone, or metabolite or analogue thereof, is in an oral dosage form. In a preferred embodiment the naltrexone, or metabolite or analogue thereof, is in the form of a tablet or capsule. The tablet or capsule may be provided as a blend of both the naltrexone product and a combination of pharmaceutically acceptable excipients.

As used herein, the term “excipient” refers to a pharmaceutically acceptable ingredient that is commonly used in pharmaceutical technology for the preparation of solid oral dosage formulations. Examples of categories of excipients include, but are not limited to, binders, disintegrants, lubricants, glidants, stabilizers, fillers, and diluents. The amount of each excipient used may vary within ranges conventional in the art. The following references which are all hereby incorporated by reference disclose techniques and excipients used to formulate oral dosage forms. See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2000).

Suitable excipients include magnesium carbonate, magnesium stearate, talc, lactose, lactose monohydrate, sugar, pectin, dextrin, starch, tragacanth, microcrystalline cellulose, methyl cellulose, sodium carboxymethyl cellulose, corn starch, colloidal anhydrous Silica, titanium dioxide, a low-melting wax, cocoa butter, and the like.

### EXAMPLES

CD3+ lymphocytes were isolated from the blood of haematologically normal volunteers. Cells placed into 25 cm3 flasks at a concentration of 1×10⁶/ml, and left to equilibrate in an incubator with a humidified atmosphere and 5% CO₂ in air at 37° C. Cells were then treated for 4 h with naltrexone (NTX) (10 uM) or low dose naltrexone (LDN) (10 nM). RNA extraction was performed using Trizol according to standard procedures, before being processed for microarray analysis according to the standard methodologies. Briefly, equal amounts of biotinylated cRNA was hybridised to the Illumina human HT12-v3 arrays for 18 h and subsequently processed according to manufacturer's instructions before scanning on an Illumina BeadArray Reader. The image data were processed using default values in GenomeStudio v2009.1 with imputation of missing data, before loading onto GeneSpring v9.0 for data normalisation and filtering. Data analyses were performed using Excel software, and the ratio of gene expression signals in treated vs. untreated calculated.

The results are shown below in Table 1.

TABLE 1

| Probe ID | Definition | Receptor | UN | NTX | LDN | NTX/UN | LDN/UN |
|---|---|---|---|---|---|---|---|
| ILMN_1715496 | 5-HT | HTR2A | 80.00 | 85.30 | 80.00 | 1.07 | 1.00 |
| | receptor 2A | | | | | | |
| | (HTR2A), | | | | | | |
| | mRNA. | | | | | | |
| ILMN_1735764 | 5-HT | HTR2B | 111.95 | 177.95 | 147.45 | 1.59 | 1.32 |
| | receptor 2B | | | | | | |
| | (HTR2B), | | | | | | |
| | mRNA. | | | | | | |
| ILMN_1801357 | 5-HT | HTR2C | 80.00 | 89.50 | 85.50 | 1.12 | 1.07 |
| | receptor 2C | | | | | | |
| | (HTR2C), | | | | | | |
| | mRNA. | | | | | | |

As can be seen from the above results, both higher dose and low dose naltrexone selectively increase the levels of mRNA for one or more of 5-HT2A, 5-HT2B or 5-HT2C receptor proteins. This effect will result in improved efficacy of treatment with agonists of a 5-HT2A, 5-HT2B or 5-HT2C receptor.

1. A method of treating a cognitive or neurological disorder in a patient, said patient having been pre-treated with naltrexone, or a metabolite thereof or an analogue selected from the group consisting of methylnaltrexone, nalmefene and nalorphine, comprising administering an agonist of a 5-hydroxytryptamine (5-HT) receptor subtype 2A, 2B or 2C (5-HT2A, 5-HT2B or 5-HT2C).

2. A method of treating a cognitive or neurological disorder in a patient, wherein said patient is also being administered or intended to be administered an agonist of a 5-hydroxytryptamine receptor subtype 2A, 2B or 2C (5-HT2A, 5-HT2B or 5-HT2C), the method comprising administering a composition comprising naltrexone, or a metabolite thereof, or an analogue selected from the group consisting of methylnaltrexone, nalmefene and nalorphine.

3. The method of claim 2, wherein the composition comprising naltrexone, or a metabolite thereof, or an analogue selected from the group consisting of methylnaltrexone, nalmefene and nalorphine, is administered separately, simultaneously or sequentially with the agonist of a 5-hydroxytryptamine receptor subtype 2A, 2B or 2C (5-HT2A, 5-HT2B or 5-HT2C).

4. The method of claim 1, wherein naltrexone, or a metabolite or analogue thereof, is in a dosage amount of 0.01 mg-50 mg.

5-6. (canceled)

7. The method of claim 1, wherein the agonist of a 5-HT2A, 5-HT2B or 5-HT2C receptor is selected from the group consisting of psilocybin, psilocin, mescaline, lysergic acid diethylamide (LSD), ketamine, salvinorin A, ibotenic acid, muscimol, N,N-dimethyltryptamine (DMT), 3,4-methylenedioxymethamphetamine (MDMA), methyldiethanolamine, also known as N-methyl diethanolamine (MDEA), 3,4-methylenedioxy amphetamine (MDA), 4-Bromo-2,5-dimethoxyphenethyl-amine (2C-B); 1-(8-Bromo-2,3,6,7-tetra-hydrobenzo[1,2-b;4,5-b′]difuran-4-yl)2-amino-ethane (2C-B-fly); 4-Ethyl-2,5-dimethoxyphenethylamine (2C-E); 4-Ethyl-thio-2,5-dimethoxy phenethylamine (2C-T-2); 4-Ethylthio-2,5-dimethoxy-amphetamine (ALEPH-2); 4-Ethylthio-2,5-dimethoxyphenylbutylamine (4C-T-2); 2,5-Dimethoxy-4-ethoxyamphetamine (MEM); 2,4,5-Trimethox-amphetamine (TMA-2); 3,4,5-Trimethoxamphetamine (TMA); 2,5-Dimethoxy-4-bromo-amphetamine (DOB); 2,5-Dimethoxy-4-iodo-amphetamine (DOI); 2,5-Dimethoxy-4-methylamphetamine (DOM); 2,5-Dimethoxy-4-ethylamphet-amine (DOET); 5-Methoxy-N,N-dimethyl-tryptamine (5-MeO-DMT); N,N-Dipropyltryptamine (DPT); 5-Methoxy-N-methyl-N-isopropyltryptamine (5-MeO-MIPT); N,N-Diisopropyltryptamine (DIPT); 5-Methoxy-N,N-diiso-propyltryptamine (5-MeO-DIPT); 6-Fluoro-N,N-dimethyltryptamine (6-fluoro-DMT); lisuride; ibogaine; cis-2a; RR-2b; SS-2c; 2-Ethyl-5-methoxy-N,N-dimethyltryptamine (EMDT), serotonin hydrochloride, m-chlorophenylbiguanide hydrochloride, N-methylquipazine dimaleate, PSEM 895, quipazine dimaleate, RS56812 hydrochloride, serotonin hydrochloride, SR7227 hydrochloride, 1-phenylbiguanide hydrochloride, cereulide, 2-methyl-5-HT, alpha-methyltryptamine, bufotenin, chlorophenylbiguanide, ibogaine, varenicline, YM-31636, or metabolites thereof and combinations thereof.

8. The method of claim 1, wherein the agonist of a 5-HT2A, 5-HT2B or 5-HT2C receptor is Psilocybin, ketamine, MDMA, LSD and the metabolites thereof.

9. The method of claim 1, wherein the cognitive or neurological disorder is Alzheimer's disease, dementia, motor neuron disease (MND), depression, psychosis, anxiety, obsessive compulsive disorder (OCD), post-traumatic stress disorder (PTSD), paranoia, panic attacks or flashbacks.

10. The method of claim 1, wherein the naltrexone, or metabolite or analogue thereof, is to be administered prior to the administration of the agonist of a 5-HT2A, 5-HT2B or 5-HT2C receptor.

11. The method of claim 10, wherein the naltrexone or a metabolite or analogue thereof, is to be administered between 1 to 4 days before administration of the agonist of a 5-HT2A, 5-HT2B or 5-HT2C receptor.

12. The method of claim 2, wherein the naltrexone, or a metabolite or analogue thereof, is in an oral dosage form.

13. The method of claim 2, wherein the naltrexone, or a metabolite or analogue thereof, is in the form of a tablet or capsule.

14-15. (canceled)

16. A method of claim 2, wherein naltrexone, or metabolite or analogue thereof, is in a dosage amount of 0.01 mg-50 mg.

17. The method of claim 2, wherein the agonist of a 5-HT2A, 5-HT2B or 5-HT2C receptor is selected from the group consisting of psilocybin, psilocin, mescaline, lysergic acid diethylamide (LSD), ketamine, salvinorin A, ibotenic acid, muscimol, N,N-dimethyltryptamine (DMT), 3,4-methylenedioxymethamphetamine (MDMA), methyldiethanolamine, also known as N-methyl diethanolamine (MDEA), 3,4-methylenedioxy amphetamine (MDA), 4-Bromo-2,5-dimethoxyphenethyl-amine (2C-B); 1-(8-Bromo-2,3,6,7-tetra-hydrobenzo[1,2-b;4,5-b′]difuran-4-yl)2-amino-ethane (2C-B-fly); 4-Ethyl-2,5-dimethoxyphenethylamine (2C-E); 4-Ethyl-thio-2,5-dimethoxy phenethylamine (2C-T-2); 4-Ethylthio-2,5-dimethoxy-amphetamine (ALEPH-2); 4-Ethylthio-2,5-dimethoxyphenylbutylamine (4C-T-2); 2,5-Dimethoxy-4-ethoxyamphetamine (MEM); 2,4,5-Trimethox-amphetamine (TMA-2); 3,4,5-Trimethoxamphetamine (TMA); 2,5-Dimethoxy-4-bromo-amphetamine (DOB); 2,5-Dimethoxy-4-iodo-amphetamine (DOI); 2,5-Dimethoxy-4-methylamphetamine (DOM); 2,5-Dimethoxy-4-ethylamphet-amine (DOET); 5-Methoxy-N,N-dimethyl-tryptamine (5-MeO-DMT); N,N-Dipropyltryptamine (DPT); 5-Methoxy-N-methyl-N-isopropyltryptamine (5-MeO-MIPT); N,N-Diisopropyltryptamine (DIPT); 5-Methoxy-N, N-diiso-propyltryptamine (5-MeO-DIPT); 6-Fluoro-N,N-dimethyltryptamine (6-fluoro-DMT); lisuride; ibogaine; cis-2a; RR-2b; SS-2c; 2-Ethyl-5-methoxy-N,N-dimethyltryptamine (EMDT), serotonin hydrochloride, m-chlorophenylbiguanide hydrochloride, N-methylquipazine dimaleate, PSEM 895, quipazine dimaleate, RS56812 hydrochloride, serotonin hydrochloride, SR7227 hydrochloride, 1-phenylbiguanide hydrochloride, cereulide, 2-methyl-5-HT, alpha-methyltryptamine, bufotenin, chlorophenylbiguanide, ibogaine, varenicline, YM-31636, or metabolites thereof and combinations thereof.

18. The method of claim 2, wherein the agonist of a 5-HT2A, 5-HT2B or 5-HT2C receptor is Psilocybin, ketamine, MDMA, LSD and the metabolites thereof.

19. The method of claim 2, wherein the cognitive or neurological disorder is Alzheimer's disease, dementia, motor neuron disease (MND), depression, psychosis, anxiety, obsessive compulsive disorder (OCD), post-traumatic stress disorder (PTSD), paranoia, panic attacks or flashbacks.

20. The method of claim 2, wherein the naltrexone, or metabolite or analogue thereof, is to be administered prior to the administration of the agonist of a 5-HT2A, 5-HT2B or 5-HT2C receptor.

21. The method of claim 20, wherein the naltrexone or a metabolite or analogue thereof, is to be administered between 1 to 4 days before administration of the agonist of a 5-HT2A, 5-HT2B or 5-HT2C receptor.
